(19) United States
(12) Patent Application Publication
(10) Pub. No.: US 2017/0275262 A1
(43) Pub. Date: Sep. 28, 2017

(54) CYCLOPROPANE DERIVATIVES IN FLAVOR AND FRAGRANCE COMPOSITIONS

(71) Applicants: Tingwei Cai, Holmdel, NJ (US); Adam P. Closson, Hoboken, NJ (US); Adam Jan Janczuk, Old Bridge, NJ (US); Ryan D. Oesterle, Jackson, NJ (US); International Flavors & Fragrances Inc., New York, NY (US)

(72) Inventors: Tingwei Cai, Holmdel, NJ (US); Adam P. Closson, Hoboken, NJ (US); Adam Jan Janczuk, Old Bridge, NJ (US); Ryan D. Oesterle, Jackson, NJ (US)

(21) Appl. No.: 15/509,026

(22) PCT Filed: Sep. 19, 2014
(86) PCT No.: PCT/US14/56536

§ 371 Date: Mar. 6, 2017

(51) Int. Cl. C07D 307/20; C07C 35/21; C07C 69/753; A23L 27/20; C11B 9/00; A23L 33/10; A23G 4/06; C07C 35/04; C07C 43/13

(52) U.S. Cl. CPC C07D 307/20; C07C 35/04; C07C 35/21; C07C 69/753; C07C 43/13; C11B 9/0076; C11B 9/003; A23L 33/10; A23G 4/06; A23L 27/20; C07C 2601/02; A23V 2002/00

(57) ABSTRACT

The present invention relates to novel cyclopropane derivatives represented by Formula I:

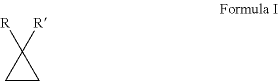

wherein R represents a hydrocarbon group containing 1-20 carbon atoms or an ester containing 1-20 carbon atoms;

wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and

wherein the composition is selected from the group consisting of a flavor composition and a fragrance composition.

### FIELD OF THE INVENTION

The present invention relates to new chemical entities and the incorporation and use of the new chemical entities as flavor and fragrance materials.

### BACKGROUND OF THE INVENTION

There is an ongoing need for flavor chemicals that enhance or provide new flavors for food preparations. There is a similar need in the fragrance industry to provide new chemicals to give perfumers and other persons the ability to create new fragrances for perfumes, colognes and personal care products. Those with skill in the art appreciate how differences in the chemical structures of the molecules can result in significant differences in the odor, notes and characteristics. The identification of structural variations and discovery of new chemicals enable the creation of new flavors and fragrances.

### SUMMARY OF THE INVENTION

The present invention is directed to the use of novel chemicals to enhance the flavor of foodstuff, chewing gums, dental and oral hygiene products and medicinal products. In addition, the present invention provides novel chemicals, and their use to enhance the fragrance of perfumes, toilet waters, colognes, personal products and the like.

More specifically, the present invention is directed to cyclopropane derivatives and a method of improving, enhancing or modifying a flavor or a fragrance composition through the addition of an olfactory acceptable amount of cyclopropane derivatives represented by Formula I set forth below:

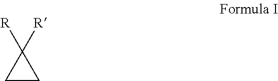

wherein R represents a hydrocarbon group containing 1-20 carbon atoms or an ester containing 1-20 carbon atoms; and

wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-0₅ heterocyclic ring.

Another embodiment of the present invention relates to a subgenus of cyclopropane derivatives represented by Formula II set forth below:

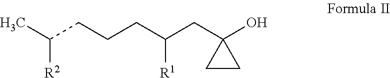

wherein R¹ and R² are each independently selected from the group consisting of H and CH₃; and

wherein the dashed line represents a single or double bond.

Another embodiment of the present invention relates to a subgenus of cyclopropane derivatives represented by Formula III set forth below:

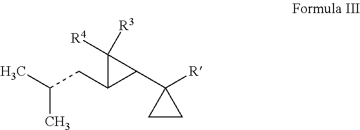

wherein R³ and R⁴ are each independently selected from the group consisting of H and CH₃;

wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and

wherein the dashed line represents a single or double bond.

Another embodiment of the invention is directed to a flavor or a fragrance composition comprising the cyclopropane derivatives provided above.

Another embodiment of the invention is directed to a composition comprising the cyclopropane derivatives provided above and a material selected from the group consisting of a foodstuff, a chewing gum, a dental product, an oral hygiene product, a medicinal product, a perfume, a cologne, toilet water, a cosmetic product, a personal care product, a fabric care product, a cleaning product and an air freshener.

A method of improving, enhancing or modifying a flavor or a fragrance composition through the addition of an olfactory acceptable amount of the cyclopropane derivatives provided above.

These and other embodiments of the present invention will be apparent by reading the following specification.

### DETAILED DESCRIPTION OF THE INVENTION

The cyclopropane derivatives represented by Formula I, II and III of the present invention are illustrated, for example, by following examples.

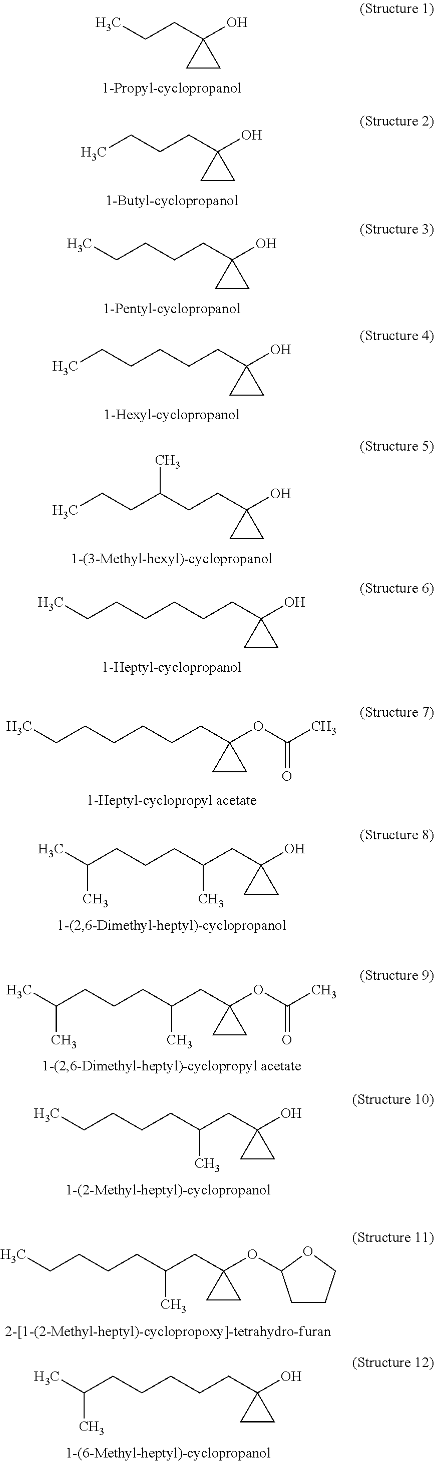

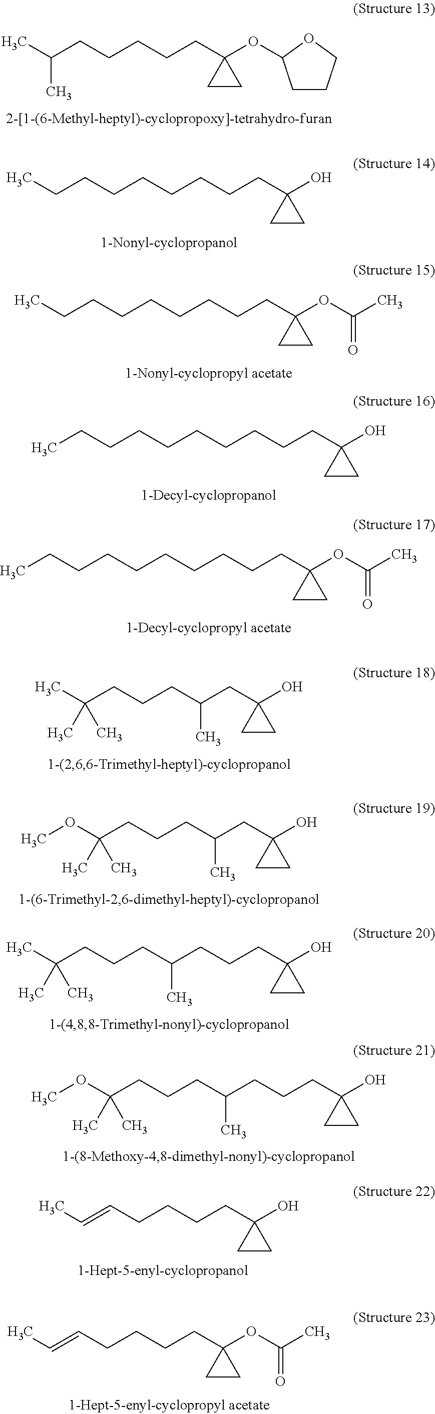

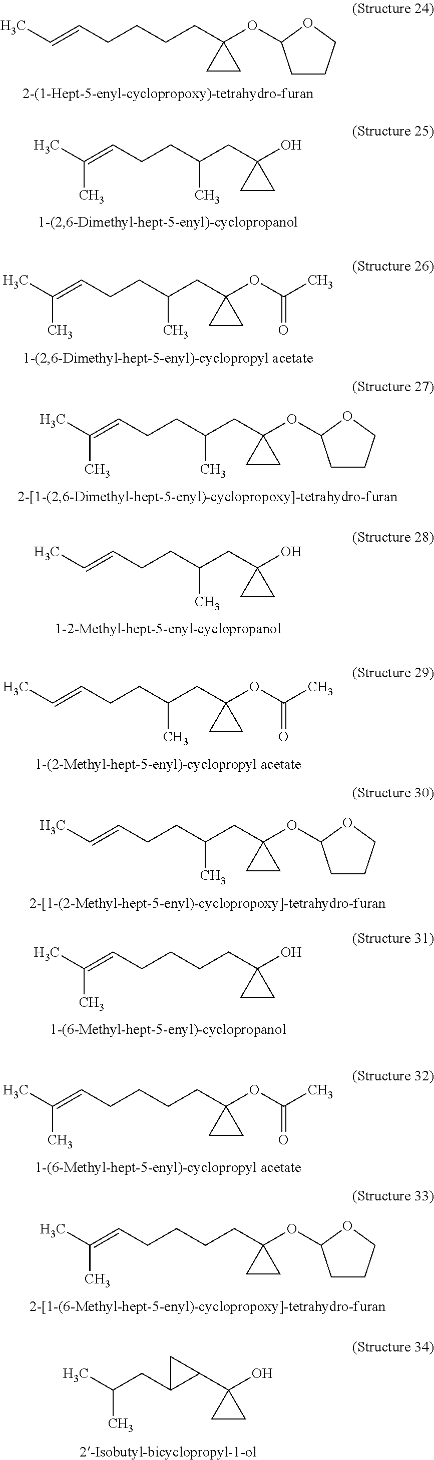

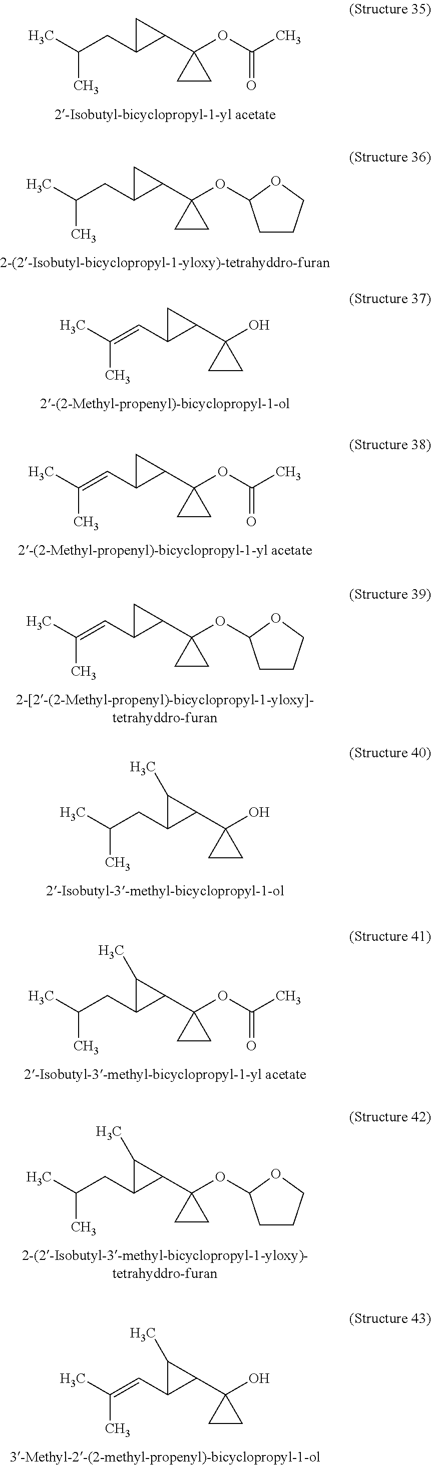

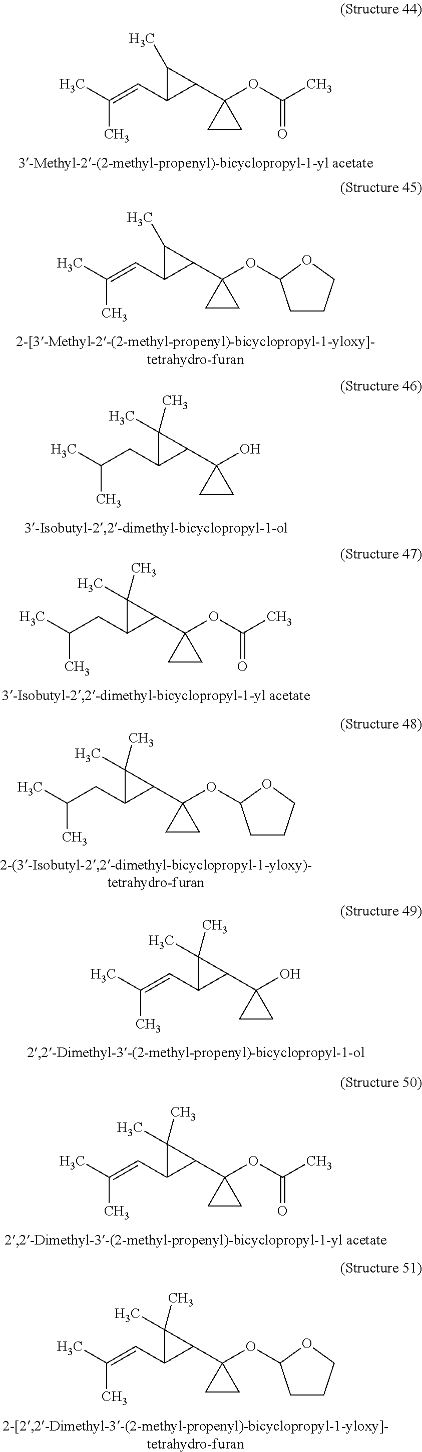

The preparation of the cyclopropanes of the present invention can be depicted by a scheme shown as follows:

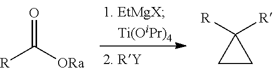

wherein R and R′ are defined as above;

Ra represents a methyl or an ethyl group;

EtMgX represents ethyl magnesium halide while X is chloride or bromide;

Ti(OⁱPr)₄ represents titanium isopropoxide; and

R′Y represents alkyl halide while Y is chloride or bromide,

with the proviso that when R′ is OH, R′Y is absent.

The above preparation is detailed in the Examples. Materials were purchased from Aldrich Chemical Company unless noted otherwise.

Those with skill in the art will recognize that some of the compounds of the present invention contain chiral centers, thereby providing a number of isomers of the claimed compounds. It is intended herein that the compounds described herein include isomeric mixtures of such compounds, as well as individual isomers that may be separated using techniques known to those having skill in the art. Suitable techniques include chromatography such as high performance liquid chromatography, referred to as HPLC, particularly silica gel chromatograph, and gas chromatography trapping known as GC trapping. Yet, commercial versions of such products are mostly offered as mixtures.

The compounds of the present invention are found to have unexpected strong and long-lasting organoleptic properties, which are shown to be advantageous for their use in augmenting or imparting taste enhancement or somatosensory effect to foodstuffs, chewing gums, dental and oral hygiene products and medicinal products by providing flavor enhancement and a preferred overall flavor profile. The present invention further relates to a process of augmenting or imparting taste or somatosensory effect to foodstuffs, chewing gums, dental and oral hygiene products and medicinal products by adding the compounds of the present invention.

When the compounds of the present invention are used in a flavoring composition, they can be combined with conventional flavoring materials or adjuvants, which are well known in the art and have been extensively described in the past. Conventional flavoring materials include saturated fatty acids, unsaturated fatty acids, amino acids; alcohols including primary and secondary alcohols; esters; carbonyl compounds including ketones; aldehydes; lactones; cyclic organic materials including benzene derivatives, acyclic compounds, heterocyclies such as furans, pyridines, pyrazines and the like; sulfur-containing compounds including thiols, sulfides, disulfides and the like; proteins; lipids; carbohydrates; so-called flavor potentiators such as monosodium glutamate; magnesium glutamate, calcium glutamate, guanylates and inosinates; natural flavoring materials such as hydrolyzates, cocoa, vanilla and caramel; essential oils and extracts such as anise oil, clove oil and the like; and artificial flavoring materials such as vanillin, ethyl vanillin and the like. Requirements for adjuvants include: (1) that they be non-reactive with the cyclopropane derivatives of the present invention; (2) that they be organoleptically compatible with the cyclopropane derivatives of the present invention, whereby the flavor of the ultimate consumable product to which the cyclopropane derivatives are added is not detrimentally affected by the use of the adjuvants; and (3) that they be ingestible acceptable and thus nontoxic or otherwise non-deleterious. In addition, other flavor materials, vehicles, stabilizers, thickeners, surface active agents, conditioners and flavor intensifiers can also be included.

The use of the compounds of the present invention is further applicable in current perfumery products, including the preparation of perfumes and colognes, the perfuming of personal care products such as soaps, shower gels, and hair care products, fabric care products, air fresheners, and cosmetic preparations. The present invention can also be used to perfume cleaning agents, such as, but not limited to detergents, dishwashing materials, scrubbing compositions, window cleaners and the like.

In these preparations, the compounds of the present invention can be used alone or in combination with other perfuming compositions, solvents, adjuvants and the like. The nature and variety of the other ingredients that can also be employed are known to those with skill in the art. Many types of fragrances can be employed in the present invention, the only limitation being the compatibility with the other components being employed. Suitable fragrances include but are not limited to fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances may also be derived from various oils, such as essential oils, or from plant materials such as peppermint, spearmint and the like.

A list of suitable fragrances is provided in U.S. Pat. No. 4,534,891, the contents of which are incorporated by reference as if set forth in its entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

The compounds of the present invention can be used in combination with a complementary fragrance compound. The term “complementary fragrance compound” as used herein is defined as a fragrance compound selected from the group consisting of 2-[(4-methylphenyl)methylene]-heptanal (Acalea), iso-amyl oxyacetic acid allylester (Allyl Amyl Glycolate), (3,3-dimethylcyclohexyl)ethyl ethyl propane-1,3-dioate (Applelide), (E/Z)-1-ethoxy-1-decene (Arctical), 2-ethyl-4-(2,2,3-trimethyl-3-cyclo-penten-1-yl)-2-buten-1-ol (Bacdanol), 2-methyl-3[(1,7,7-trimethylbicyclo[2.2.1]hept-2-yl)oxy] exo-1-propanol (Bornafix), 1,2,3 ,5,6,7-hexahydro-1,1,2,3,3 -pentamethyl-4H-inden-4-one (Cashmeran), trimethylcyclopentenylmethyloxabicyclooctane (Cassiffix), 1,1-dimethoxy-3,7-dimethyl-2,6-octadiene (Citral DMA), 3 ,7-dimethyl-6-octen-1-ol (Citronellol), 3A,4,5,6,7,7A-hexahydro-4,7-methano-1H-inden-5/6-yl acetate (Cyclacet), 3A,4,5,6,7,7A-hexahydro-4,7-methano-1H-inden-5/6-yl propinoate (Cyclaprop), 3A,4,5,6,7,7A-hexahydro-4,7-methano-1G-inden-5/6-yl butyrate (Cyclobutanate), 1-(2,6,6-trimethyl-3 -cyclohexen-1-yl)-2-buten-1-one (Delta Damascone), 3-(4-ethylphenyl)-2,2-dimethyl propanenitrile (Fleuranil), 3-(O/P-ethylphenyl) 2,2-dimethyl propionaldehyde (Floralozone), tetrahydro-4-methyl-2-(2-methylpropyl)-2H-pyran-4-ol (Floriffol), 1,3,4,6,7,8-hexahydro-4,6,6,7,8,8-hexamethylcyclopenta-gamma-2-benzopyran (Galaxolide), 1-(5 ,5 -dimethyl-1-cyclohexen-1-yl)pent-4-en-1-one (Galbascone), E/Z-3 ,7-dimethyl-2,6-octadien-1-yl acetate (Geranyl Acetate), α-methyl-1,3-benzodioxole-5-propanal (Helional), 1-(2,6,6-trimethyl-2-cyclohexen-1-yl)-1,6-heptadien-3 -one (Hexalon), (Z)-3-hexenyl-2-hydroxybenzoate (Hexenyl Salicylate, CIS-3), 4-(2,6,6-trimethyl-2-cyclohexen- 1-yl)-3-buten-2-one (Ionone α), 1-(1,2,3,4,5,6,7,8-octahydro-2,3,8,8-tetramethyl-2-naphthalenyl)-ethan-1-one (Iso E Super), methyl 3-oxo-2-pentylcyclopentaneacetate (Kharismal), 2,2,4-trimethyl-4-phenyl-butanenitrile (Khusinil), 3,4,5,6,6-pentamethylhept-3-en-2-one (Koavone), 3/4-(4-hydroxy-4-methyl-pentyl) cyclohexene-1-carboxaldehyde (Lyral), 3-methyl-4-(2,6,6-trimethyl-2-cyclohexen-1-yl)-3-buten-2-one (Methyl Ionone γ), 1-(2,6,6-trimethyl-2-cyclohexen-1-yl) pent-1-en-3-one (Methyl Ionone α Extra, Methyl Ionone N), 3-methyl-4-phenylbutan-2-ol (Muguesia), cyclopentadec-4-en-1-one (Musk Z4), 3,3,4,5,5-pentamethyl-11,13-dioxatricyclo[7.4.0.0<2,6>]tridec-2(6)-ene (Nebulone), 3,7-dimethyl-2,6-octadien-1-yl acetate (Neryl Acetate), 3,7-dimethyl-1,3,6-octatriene (Ocimene), ortho-tolylethanol (Peomosa), 3-methyl-5-phenylpentanol (Phenoxanol), 1-methyl-4-(4-methyl-3 -pentenyl) cyclohex-3-ene-1-carboxaldehyde (Precyclemone B), 4-methyl-8-methylene-2-adamantanol (Prismantol), 2-ethyl-4-(2,2,3 -trimethyl-3 -cyclopenten-1-yl)-2-buten-1-ol (Sanjinol), 2-methyl-4-(2,2,3 -trimethyl-3 -cyclopenten-1-yl)-2-buten-1-ol (Santaliff), Terpineol, 2,4-dimethyl-3-cyclohexene- 1-carboxaldehyde (Triplal), decahydro-2,6,6,7,8,8-hexamethyl-2H-indeno[4,5-B]furan (Trisamber), 2-tert-butylcyclohexyl acetate (Verdox), 4-tert-butylcyclohexyL acetate (Vertenex), acetyl cedrene (Vertofix), 3,6/4,6-dimethylcyclohex-3-ene-1-carboxaldehyde (Vertoliff), and (3Z)-1-[(2-methyl-2-propenyl)oxy]-3-hexene (Vivaldie).

The term “hydrocarbon group” means a chemical group that contains only hydrogen and carbon atoms. The hydrocarbon group of the present invention can be a straight, branched and/or cyclic, saturated or unsaturated group.

The term “foodstuff' as used herein includes both solid and liquid ingestible materials for man or animals, which materials usually do, but need not, have nutritional value. Thus, foodstuffs include meats, gravies, soups, convenience foods, malt, alcoholic and other beverages, milk and dairy products, seafood, including fish, crustaceans, mollusks and the like, candies, vegetables, cereals, soft drinks, snacks, dog and cat foods, other veterinary products and the like.

The terms “flavor composition” and “flavor formulation” mean the same and refer to a consumer composition that produces a pleasant or desired flavor. The flavor composition contains a compound or a mixture of compounds. The flavor composition of the present invention is a consumer composition comprising a compound of the present invention.

The terms “fragrance composition”, “fragrance formulation” and “perfume composition” mean the same and refer to a consumer composition that is a mixture of compounds including, for example, alcohols, aldehydes, ketones, esters, ethers, lactones, nitriles, natural oils, synthetic oils, and mercaptans, which are admixed so that the combined odors of the individual components produce a pleasant or desired fragrance. The fragrance composition of the present invention is a consumer composition comprising a compound of the present invention. The fragrance composition of the present invention comprises a compound of the present invention and further a complementary fragrance compound as defined above.

The term “fragrance product” means a consumer product containing a fragrance ingredient that adds fragrance or masks malodor. Fragrance products may include, for example, perfumes, colognes, bar soaps, liquid soaps, shower gels, foam baths, cosmetics, skin care products such as creams, lotions and shaving products, hair care products for shampooing, rinsing, conditioning, bleaching, coloring, dyeing and styling, deodorants and antiperspirants, feminine care products such as tampons and feminine napkins, baby care products such as diapers, bibs and wipes, family care products such as bath tissues, facial tissues, paper handkerchiefs or paper towels, fabric products such as fabric softeners and fresheners, air care products such as air fresheners and fragrance delivery systems, cosmetic preparations, cleaning agents and disinfectants such as detergents, dishwashing materials, scrubbing compositions, glass and metal cleaners such as window cleaners, countertop cleaners, floor and carpet cleaners, toilet cleaners and bleach additives, washing agents such as all-purpose, heavy duty, and hand washing or fine fabric washing agents including laundry detergents and rinse additives, dental and oral hygiene products such as toothpastes, tooth gels, dental flosses, denture cleansers, denture adhesives, dentifrices, tooth whitening and mouthwashes, health care and nutritional products and food products such as snack and beverage products. The fragrance product of the present invention is a consumer product that contains a compound of the present invention. The fragrance product of the present invention contains a compound of the present invention and further a complementary fragrance compound as defined above.

The term “improving” is understood to mean raising a flavor or fragrance composition to a more desirable character. The term “enhancing” is understood to mean making the flavor or fragrance composition greater in effectiveness or providing the flavor or fragrance composition with an improved character. The term “modifying” is understood to mean providing the flavor or fragrance composition with a change in character.

The term “olfactory acceptable amount” is understood to mean the amount of a compound in a flavor or fragrance formulation, wherein the compound will contribute its individual olfactory characteristics. However, the olfactory effect of the flavor or fragrance formulation will be the sum of effect of each of the flavor or fragrance ingredients. Thus, the compound of the present invention can be used to improve or enhance the aroma characteristics of the flavor or fragrance formulation, or by modifying the olfactory reaction contributed by other ingredients in the formulation. The olfactory acceptable amount may vary depending on many factors including other ingredients, their relative amounts and the olfactory effect that is desired.

Generally, the olfactory acceptable amount of the cyclopropane derivatives employed in a flavor composition is greater than about 0.1 parts per billion by weight, preferably from about 1 part per billion to about 500 parts per million by weight, more preferably from about 10 parts per billion to about 100 parts per million by weight, even more preferably from about 100 parts per billion to about 50 parts per million by weight. The olfactory acceptable amount of the cyclopropane derivatives employed in a fragrance composition varies from about 0.005 to about 70 weight percent, preferably from 0.005 to about 50 weight percent, more preferably from about 0.5 to about 25 weight percent, and even more preferably from about 1 to about 10 weight percent. Those with skill in the art will be able to employ the desired amount to provide desired flavor or fragrance effect and intensity. In addition to the compounds of the present invention, other materials can also be used in conjunction with the flavor or fragrance composition to encapsulate and/or deliver the flavor or fragrance. Some well-known materials are, for example, but not limited to, polymers, oligomers, other non-polymers such as surfactants, emulsifiers, lipids including fats, waxes and phospholipids, organic oils, mineral oils, petrolatum, natural oils, perfume fixatives, fibers, starches, sugars and solid surface materials such as zeolite and silica.

The following are provided as specific embodiments of the present invention. Other modifications of this invention will be readily apparent to those skilled in the art. Such modifications are understood to be within the scope of this invention. As used herein all percentages are weight percent unless otherwise noted, ppb is understood to stand for parts per billion, ppm is understood to stand for parts per million, L is understood to be liter, mL is understood to be milliliter, Kg is understood to be kilogram, g is understood to be gram, mol is understood to be mole and M is understood to be molar. IFF as used in the examples is understood to mean International Flavors & Fragrances Inc., New York, N.Y., USA.

### EXAMPLE I

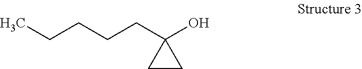

Preparation of 1-Pentyl-cyclopropanol (Structure 3): A 5L round bottom flask was charged with hexanoic acid methyl ester (CH₃(CH₂)₄COOCH₃) (195 g, 1.5 mol) and tetrahydrofuran (THF) (1 L). The reaction mixture was then placed under nitrogen (N₂) followed by the addition of titanium isopropoxide (Ti(OPr)₄) (44 mL, 0.15 mol). The resulting mixture was cooled to 0° C. using an ice water bath and stirred. A freshly prepared solution of ethyl magnesium bromide (C₂H₅MgBr) (3 M in THF, 1 L, 3.0 mol) was subsequently added dropwise while the temperature was maintained below 30° C. After the addition of ethyl magnesium bromide was complete, the reaction mixture was quenched with sulfuric acid solution (H₂SO₄) (10%, 1 L) on ice and then loaded into a separatory funnel. The resulting layers were separated and the organic layers were washed twice with saturated sodium carbonate solution (Na₂CO₃) followed by in vacuo removal of solvent. Further fractional distillation afforded 1-pentyl-cyclopropanol (137 g, 1.06 mol).

¹H NMR (CDCl₃, 500 MHz): 1.83 (br s, 1 H), 1.43-1.62 (m, 4 H), 1.27-1.38 (m, 4 H), 0.90 (t, J=6.7 Hz, 3 H), 0.68-0.77 (m, 2 H), 0.39-0.48 (m, 2 H)

1-Pentyl-cyclopropanol was described as having fresh, green, earthy and mushroom organoleptic properties with fatty, musty and metallic characters.

### EXAMPLE II

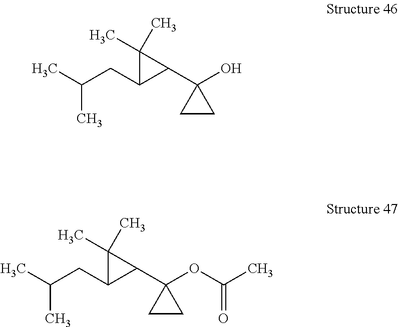 [SMILES: CC(C)CC1C(C2(O)CC2)C1(C)C ; CC(=O)OC1(C2C(CC(C)C)C2(C)C)CC1]

Preparation of 3′-Isobutyl-2′,2′-dimethyl-bicyclopropyl-1-ol (Structure 46) and 3′-Isobutyl-2′,2′-dimethyl-bicyclopropyl-1-yl Acetate (Structure 47): A 3L round bottom flask was charged with dihydro ethyl chrysanthemumate (C₁₂H₂₂O₂) (146 g, 0.73 mol), magnesium (Mg) (45 g, 1.8 mol), titanium isopropoxide (42 g, 0.14 mol) and tetrahydrofuran (1 L). Ethyl bromide (EtBr) (201 g, 1.8 mol) was then fed dropwise at room temperature. After the reaction initiated, the reaction temperature was maintained at 35° C. until the reaction was complete. The reaction mixture was then quenched with sulfuric acid solution (10%, 1 L) on ice. The resulting layers were separated and the aqueous layer was extracted using toluene (300 mL). The organic layers were combined and washed with sodium carbonate solution followed by in vacuo removal of solvent. The crude product 3′-isobutyl-2′,2′-dimethyl-bicyclopropyl-1-ol (134 g, 0.73 mol) was obtained and used in following steps without further purification.

The solution of 3′-isobutyl-2′,2′-dimethyl-bicyclopropyl-1-ol (134g, 0.73 mol) in toluene (500 mL) was stirred with acetic anhydride ((CH₃CO)₂O) (112 g, 1.1 mol) and dimethylaminopyridine ((CH₃)₂NC₅H₄N) (1 g, 0.01 mol) at 80° C. When gas chromatography (GC) analysis showed the reaction was complete, the reaction mixture was quenched with water and then heated to 80° C. After stirring for another hour, the reaction mixture was cooled to room temperature and then loaded into a separatory funnel. The resulting layers were separated and the organic layers were washed with sodium carbonate solution. Further fractional distillation afforded product 3′-isobutyl-2′,2′-dimethyl-bicyclopropyl-1-yl acetate (124 g, 0.55 mol).

3′-Isobutyl-2′,2′-dimethyl-bicyclopropyl-1-ol (Structure 46):

¹H NMR (CDCl₃, 500 MHz): 2.21 (bs, OH), 1.45-1.58 (m, 1 H), 1.22 (s, 3 H), 1.17-1.20 (m, 1 H), 1.07 (s, 3 H),0.82-0.94 (m, 6 H), 0.25-0.82 (m, 7 H).

3′-Isobutyl-2′,2′-dimethyl-bicyclopropyl-1-yl acetate (Structure 47):

¹H NMR (CDCl₃, 500 MHz): 1.98 (s, 3 H), 1.50 (m, 1 H), 1.19 (s, 3 H), 1.08-1.11 (m, 2 H) 1.00 (s, 3 H), 0.73-0.94 (m, 10 H), 0.58 (m, 1 H), 0.364 (m, 1 H).

3′-Isobutyl-2′,2′-dimethyl-bicyclopropyl-1-yl acetate was described as having herbaceous, earthy, woody, fruity, spicy and bitter organoleptic properties.

### EXAMPLE III

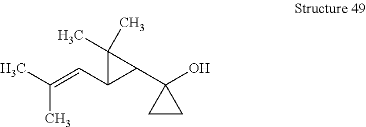

Preparation of 2′,2′-Dimethyl-3′-(2-methyl-propenyl)-bicyclopropyl-1-ol (Structure 49): A 3L round bottom flask was charged with ethyl chrysanthemumate (C₁₂H₂₀O₂) (250 g, 1.27 mol), magnesium (77 g, 3.1 mol) and tetrahydrofuran (1 L). The reaction mixture was then placed under nitrogen (N₂) followed by the addition of titanium isopropoxide (72 g, 0.25 mol). Ethyl bromide (346 g, 3.1 mol) was fed dropwise at room temperature. The reaction was exothermic and the temperature increased to 45° C. After the addition of ethyl bromide was complete, the reaction mixture was quenched with sulfuric acid solution (10%, 1 L) on ice. The resulting layers were separated and the aqueous layer was extracted using toluene (300 mL). The organic layers were combined and washed with sodium carbonate solution. Further fractional distillation afforded 2′,2′-dimethyl-3′-(2-methyl-propenyl)-bicyclopropyl-l-ol (103 g, 0.57 mol).

¹H NMR (CDCl₃, 500 MHz): 5.17 (d, J=7.98 Hz, 1 H), 1.73 (s, 3 H), 1.71 (s, 3 H), 1.24 (s, 3 H), 1.11 (s, 3 H), 0.44-0.99 (m, 6 H).

2′,2′-Dimethyl-3′-(2-methyl-propenyl)-bicyclopropyl-1-ol was described as having green, floral, musky, bitter and musty organoleptic properties.

### EXAMPLE IV

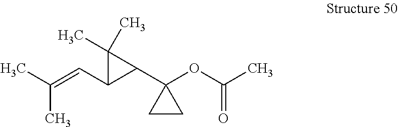 [SMILES: CC(=O)OC1(C2C(C=C(C)C)C2(C)C)CC1]

Preparation of 2′,2′-Dimethyl-3′-(2-methyl-propenyl)-bicyclopropyl-1-yl Acetate (Structure 50): A 3L round bottom flask was charged with 2′,2′-dimethyl-3′-(2-methyl-propenyl)-bicyclopropyl-1-ol (obtained above in EXAMPLE III) (265 g, 1.47 mol), acetic anhydride (225 g, 2.06 mol) and dimethylaminopyridine (1 g, 0.01 mol). The resulting mixture was stirred at room temperature. When gas chromatography analysis showed the reaction was complete, the reaction mixture was quenched with water and then heated to 80° C. After stirring for another hour, the reaction mixture was cooled to room temperature and then loaded into a separatory funnel. The resulting layers were separated and the organic layers were washed with sodium carbonate solution. Further fractional distillation afforded product 2′,2′-dimethyl-3′-(2-methyl-propenyl)-bicyclopropyl-1-yl acetate (140 g, 0.63 mol).

¹H NMR (CDCl₃, 500 MHz): 4.79 (d, J=8.83 Hz, 1 H), 1.99 (s, 3 H), 1.67 (s, 3 H), 1.23 (s, 3 H), 1.01 (s, 3 H), 0.50-0.97 (m, 6 H).

2′2′-Dimethyl-3′-(2-methyl-propenyl)-bicyclopropyl-1-yl acetate was described as having green, herbaceous, citrus and honey organoleptic properties.

### EXAMPLE V

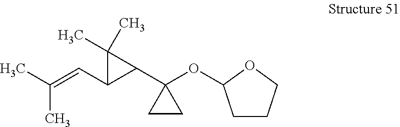 [SMILES: CC(C)=CC1C(C2(OC3CCCO3)CC2)C1(C)C]

Preparation of 2-[2′,2′-Dimethyl-3′-(2-methyl-propenyl)-bicyclopropyl-1-yloxy]-tetrahydro-furan (Structure 51): A 3L round bottom flask was charged with 2′,2′-dimethyl-3′-(2-methyl-propenyl)-bicyclopropyl-1-ol (obtained above in EXAMPLE III) (230 g, 1.2 mol) and toluene (500 mL). p-Toluenesulfonic acid was added as a reaction catalyst. 2,3-Dihydrofuran (89 g, 1.2 mol) was then fed dropwise at room temperature, resulting an exothermic reaction. When gas chromatography analysis showed the reaction was complete, the reaction mixture was quenched with sodium methoxide solution (CH₃ONa) (10 g, 0.33 mol) and concentrated. Short path distillation provided a crude product, which was further fractionated to afford product 2-[2′,2′-dimethyl-3′-(2-methyl-propenyl)-bicyclopropyl-1-yloxy]-tetrahydro-furan (23 g, 0.104 mol).

¹H NMR (CDCl₃, 500 MHz): 5.54 (d, J=4.4 Hz, 0.45 H), 5.37(d, J=4.4 Hz, 0.55 H), 4.81 (d, J=5.14 Hz, 1 H), 3.78-3.96 (m, 2 H), 1.72-2.03 (m, 4 H), 1.67(s, 3 H), 1.61 (s, 3 H), 1.23 (s, 3 H), 1.07 (s, 3 H), 0.42-0.99 (m, 6 H).

2-[2′,2′-Dimethyl-3′-(2-methyl-propenyl)-bicyclopropyl-1-yloxy]-tetrahydro-furan was described as having earthy, mushroom and metallic organoleptic properties.

### EXAMPLE VI

Following cyclopropane derivatives were similarly prepared.

1-Hexyl-cyclopropanol (Structure 4):

¹H NMR (CDCl₃, 500 MHz): 1.87 (br s, 1 H), 1.45-1.59 (m, 4 H), 1.25-1.37 (m, 6 H), 0.89 (t, J=6.4 Hz, 3 H), 0.69-0.75 (m, 2 H), 0.41-0.45 (m, 2 H)

1-Hexyl-cyclopropanol was described as having fresh, green, earthy, mushroom and floral organoleptic properties.

1-(3-Methyl-hexyl)-cyclopropanol (Structure 5):

¹H NMR (CDCl₃, 400 MHz): 1.91 (br s, 1 H), 1.01-1.62 (m, 9 H), 0.88 (t, J=7.1 Hz, 3 H), 0.87 (d, J=5.5 Hz, 3 H), 0.66-0.79 (m, 2 H), 0.37-0.50 (m, 2 H)

1-(3-Methyl-hexyl)-cyclopropanol was described as having fresh, green, earthy, mushroom, floral, fruity and sweet organoleptic properties.

1-Heptyl-cyclopropanol (Structure 6):

¹H NMR (CDCl₃, 500 MHz): 1.77 (br s, 1 H), 1.43-1.58 (m, 4 H), 1.22-1.36 (m, 8 H), 0.89 (t, J=6.5 Hz, 3 H), 0.69-0.75 (m, 2 H), 0.41-0.46 (m, 2 H)

1-Heptyl-cyclopropanol was described as having green, jammy, floral and spicy organoleptic properties.

1-Decyl-cyclopropanol (Structure 16):

¹H NMR (CDCl₃, 500 MHz): 1.78 (s, 1 H), 1.46-1.58 (m, 4 H), 1.23-1.35 (m, 14 H), 0.88 (t, J=6.9 Hz, 3 H), 0.70-0.75 (m, 2 H), 0.42-0.46 (m, 2 H)

1-Decyl-cyclopropanol was described as having chemical, rubbery and fatty organoleptic properties.

1-Decyl-cyclopropyl Acetate (Structure 17):

¹H NMR (CDC1₃, 500 MHz): 1.98 (s, 3 H), 1.71-1.77 (m, 2 H), 1.34-1.47 (m, 2 H), 1.23-1.31 (m, 14 H), 0.88 (t, J=6.8 Hz, 3 H), 0.78-0.85 (m, 2 H), 0.62-0.66 (m, 2 H)

1-Decyl-cyclopropyl acetate was described as having mushroom and floral organoleptic properties with weak and metallic characters.

1-(8-Methoxy-4,8-dimethyl-nonyl)-cyclopropanol (Structure 21):

¹H NMR (CDC1₃, 400 MHz): 3.17 (s, 3 H), 2.35 (br s, 1 H), 1.05-1.62 (m, 13 H), 1.14 (s, 6 H), 0.88 (d, J=6.5 Hz, 3 H), 0.65-0.78 (m, 2 H), 0.35-0.50 (m, 2 H)

1-(8-Methoxy-4,8-dimethyl-nonyl)-cyclopropanol was described as having green, earthy woody, animalic, bitter and weak organoleptic properties.

1-(2,6-Dimethyl-hept-5-enyl)-cyclopropanol (Structure 25):

¹H NMR (CDCl₃, 400 MHz): 5.11 (t, J=6.9 Hz, 1 H), 1.90-2.07 (m, 3 H), 1.79-1.90 (m, 1 H), 1.66-1.75 (m, 1 H), 1.68 (s, 3 H), 1.61 (s, 3 H), 1.35-1.53 (m, 1 H), 1.11-1.33 (m, 2 H), 1.00 (d, J=6.5 Hz, 3 H), 0.69-0.88 (m, 2 H), 0.36-0.50 (m, 2 H)

1-(2,6-Dimethyl-hept-5-enyl)-cyclopropanol was described as having fresh, citrus, floral, fruity and spicy organoleptic properties.

What is claimed is:

1. A composition comprising an olfactory acceptable amount of a compound of Formula I: 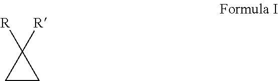
wherein R represents a hydrocarbon group containing 1-20 carbon atoms or an ester containing 1-20 carbon atoms;
wherein R′represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and
wherein the composition is selected from the group consisting of a flavor composition and a fragrance composition.

2. The composition of claim 1, wherein the compound is selected from the group consisting of Formula II and Formula III: 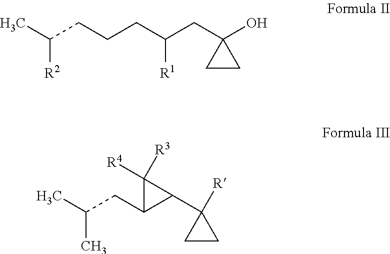
wherein R′, R², R³ and R⁴ are each independently selected from the group consisting of H and CH₃;
wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and
wherein the dashed line represents a single or double bond.

3. The composition of claim 2, wherein the compound is selected from the group consisting of:
1-heptyl-cyclopropanol;
1-(2,6-dimethyl-heptyl)-cyclopropanol;
1-(2-methyl-heptyl)-cyclopropanol;
1-(6-methyl-heptyl)-cyclopropanol;
1-hept-5 -enyl-cyclopropanol;
1-(2,6-dimethyl-hept-5-enyl)-cyclopropanol;
1-2-methyl-hept-5 -enyl-cyclopropanol;
1-(6-methyl-hept-5-enyl)-cyclopropanol;
2′-isobutyl-bicyclopropyl-1-ol;
2′-isobutyl-bicyclopropyl-1-yl acetate;
2-(2′-isobutyl-bicyclopropyl-1-yloxy)-tetrahydro-furan;
2′-(2-methyl-propenyl)-bicyclopropyl-1-ol;
2′-(2-Methyl-propenyl)-bicyclopropyl-1-yl acetate;
2-[2′-(2-methyl-propenyl)-bicyclopropyl-1-yloxy]-tetrahydro-furan;
2′-Isobutyl-3′-methyl-bicyclopropyl-1-ol;
2′-Isobutyl-3′-methyl-bicyclopropyl-1-yl acetate;
2-(2′-Isobutyl-3′-methyl-bicyclopropyl-1-yloxy)-tetrahydro-furan;
3-Methyl-2′-(2-methyl-propenyl)-bicyclopropyl-1-ol;
3-Methyl-2′-(2-methyl-propenyl)-bicyclopropyl-1-yl acetate;
2-[3-Methyl-2′-(2-methyl-propenyl)-bicyclopropyl-1-yloxy]-tetrahydro-furan;
3-Isobutyl-2′,2′-dimethyl-bicyclopropyl-1-ol;
3-Isobutyl-2′,2′-dimethyl-bicyclopropyl-1-yl acetate;
2-(3-Isobutyl-2′,2′-dimethyl-bicyclopropyl-1-yloxy)-tetrahydro-furan;
2′,2′-Dimethyl-3′-(2-methyl-propenyl)-bicyclopropyl-1-ol;
2′,2′-Dimethyl-3′-(2-methyl-propenyl)-bicyclopropyl-1-yl acetate; and
2[2′,2′-Dimethyl-3′-(2-methyl-propenyl)-bicyclopropyl-1-yloxy]-tetrahydro-furan.

4. The composition of claim 1, wherein the composition is the flavor composition further comprising a material selected from the group consisting of a foodstuff, a chewing gum, a dental product, an oral hygiene product and a medicinal product.

5. The composition of claim 4, wherein the olfactory acceptable amount is greater than about 0.1 parts per billion by weight of the composition.

6. The composition of claim 4, wherein the olfactory acceptable amount is from about 1 part per billion to about 500 parts per million by weight of the composition.

7. The composition of claim 4, wherein the olfactory acceptable amount is from about 10 parts per billion to about 100 parts per million by weight of the composition.

8. The composition of claim 1, wherein the composition is the fragrance composition further comprising a material selected from the group consisting of a perfume, a cologne, toilet water, a cosmetic product, a personal care product, a fabric care product, a cleaning product and an air freshener.

9. The composition of claim 8, wherein the olfactory acceptable amount is from about 0.005 to about 50 weight percent of the composition.

10. The composition of claim 8, wherein the olfactory acceptable amount is from about 0.5 to about 25 weight percent of the composition.

11. The composition of claim 8, wherein the olfactory acceptable amount is from about 1 to about 10 weight percent of the composition.

12. A method of improving, enhancing or modifying a composition through the addition of an olfactory acceptable amount of a compound of Formula I: 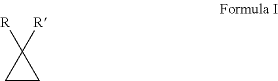
wherein R represents a hydrocarbon group containing 1-20 carbon atoms or an ester containing 1-20 carbon atoms;
wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and
wherein the composition is selected from the group consisting of a flavor composition and a fragrance composition.

13. The method of claim 12, wherein the compound is selected from the group consisting of Formula II and Formula III; 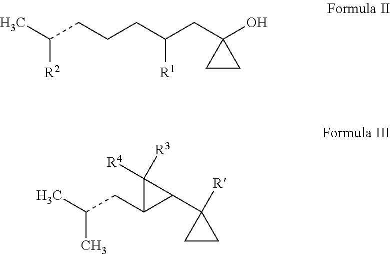
wherein R¹, R², R³ and R⁴ are each independently selected from the group consisting of H and CH₃;
wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and
wherein the dashed line represents a single or double bond.

14. The method of claim 13, wherein the compound is selected from the group consisting of:
1-heptyl-cyclopropanol;
1-(2,6-dimethyl-heptyl)-cyclopropanol;
1-(2-methyl-heptyl)-cyclopropanol;
1-(6-methyl-heptyl)-cyclopropanol;
1-hept-5-enyl-cyclopropanol;
1-(2,6-dimethyl-hept-5-enyl)-cyclopropanol;
1-2-methyl-hept-5-enyl-cyclopropanol;
1-(6-methyl-hept-5-enyl)-cyclopropanol;
2′-isobutyl-bicyclopropyl-1-ol;
2′-isobutyl-bicyclopropyl-1-yl acetate;
2-(2′-isobutyl-bicyclopropyl-1-yloxy)-tetrahydro-furan;
2-′-(2-methyl-propenyl)-bicyclopropyl-1-ol;
2′-(2-Methyl-propenyl)-bicyclopropyl-1-yl acetate;
2-[2′(2-methyl-propenyl)-bicyclopropyl-1-yloxy]-tetrahydro-furan;
2′-Isobutyl-3-methyl-bicyclopropyl-1-ol;
2′-Isobutyl-3-methyl-bicyclopropyl-1-yl acetate;
2-(2′-Isobutyl-3′-methyl-bicyclopropyl-1-yloxy)-tetrahydro-furan;
3-Methyl-2′-(2-methyl-propenyl)-bicyclopropyl-1-ol;
3-Methyl-2′-(2-methyl-propenyl)-bicyclopropyl-1-yl acetate;
2-[3′-Methyl-2′-(2-methyl-propenyl)-bicyclopropyl-1-yloxy]-tetrahydro-furan;
3-Isobutyl-2′,2′-dimethyl-bicyclopropyl-1-ol;
3-Isobutyl-2′,2′-dimethyl-bicyclopropyl-1-yl acetate;
2-(3′-Isobutyl-2′,2′-dimethyl-bicyclopropyl-1-yloxy)-tetrahydro-furan;
2′,2′-Dimethyl-3-(2-methyl-propenyl)-bicyclopropyl-1-ol;
2′,2′-Dimethyl-3-(2-methyl-propenyl)-bicyclopropyl-1-yl acetate; and
2-[2′,2′-Dimethyl-3-(2-methyl-propenyl)-bicyclopropyl-1-yloxy]-tetrahydro-furan.

15. The method of claim 12, wherein the composition is the flavor composition; and
wherein the olfactory acceptable amount is greater than about 0.1 parts per billion by weight of the composition.

16. The method of claim 12, wherein the composition is the flavor composition; and
wherein the olfactory acceptable amount is from about 1 part per billion to about 500 parts per million by weight of the composition.

17. The method of claim 12, wherein the composition is the flavor composition; and
wherein the olfactory acceptable amount is from about 10 parts per billion to about 100 parts per million by weight of the composition.

18. The method of claim 12, wherein the composition is the fragrance composition; and
wherein the olfactory acceptable amount is from about 0.005 to about 50 weight percent of the composition.

19. A compound selected from the group consisting of Formula II and Formula III; 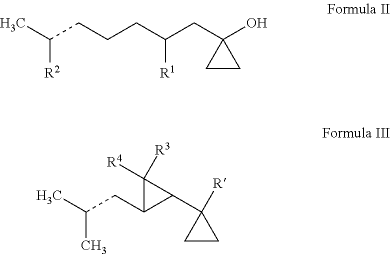
wherein R¹, R², R³ and R⁴ are each independently selected from the group consisting of H and CH₃;
wherein R′ represents a C₁-C₆ acyclic carboxylic acid ester, a C₄-C₆ cyclic carboxylic acid ester or —OR″, and R″ is selected from the group consisting of H, a C₁-C₆ acyclic hydrocarbon group, a C₃-C₆ carbocyclic ring and a C₄-C₅ heterocyclic ring; and
wherein the dashed line represents a single or double bond.

20. The compound of claim 15, wherein the compound selected from the group consisting of:
1-heptyl-cyclopropanol;
1-(2,6-dimethyl-heptyl)-cyclopropanol;
1-(2-methyl-heptyl)-cyclopropanol;
1-(6-methyl-heptyl)-cyclopropanol;
1-hept-5-enyl-cyclopropanol;
1-(2,6-dimethyl-hept-5-enyl)-cyclopropanol;
1-2-methyl-hept-5 -enyl-cyclopropanol;
1-(6-methyl-hept-5 -enyl)-cyclopropanol;
2′-isobutyl-bicyclopropyl-1-ol;
2′-isobutyl-bicyclopropyl-1-yl acetate;
2-(2′-isobutyl-bicyclopropyl-1-yloxy)-tetrahydro-furan;
2′-(2-methyl-propenyl)-bicyclopropyl-1-ol;
2′-(2-Methyl-propenyl)-bicyclopropyl-1-yl acetate;
2′-[2′(2-methyl-propenyl)-bicyclopropyl-1-yloxy]-tetrahydro-furan;
2′-Isobutyl-3′-methyl-bicyclopropyl-1-ol;
2′-Isobutyl-3′-methyl-bicyclopropyl-1-yl acetate;
2-(2′-Isobutyl-3′-methyl-bicyclopropyl-1-yloxy)-tetrahydro-furan;
3-Methyl-2′-(2-methyl-propenyl)-bicyclopropyl-1-ol;
3-Methyl-2′-(2-methyl-propenyl)-bicyclopropyl-1-yl acetate;
2-[3-Methyl-2′-(2-methyl-propenyl)-bicyclopropyl-1-yloxy]-tetrahydro-furan;
3-Isobutyl-2′,2′-dimethyl-bicyclopropyl-1-ol;
3-Isobutyl-2′,2′-dimethyl-bicyclopropyl-1-yl acetate;
2-(3′-Isobutyl-2′,2′-dimethyl-bicyclopropyl-1-yloxy)-tetrahydro-furan;
2′,2′-Dimethyl-3′-(2-methyl-propenyl)-bicyclopropyl-1-ol;
2′,2′-Dimethyl-3′-(2-methyl-propenyl)-bicyclopropyl-1-yl acetate; and
2-[2′,2′-Dimethyl-3-(2-methyl-propenyl)-bicyclopropyl-1-yloxyl-tetrahydro-furan.
